(19) United States
(12) Patent Application Publication
(10) Pub. No.: US 2007/0060638 A1
(43) Pub. Date: Mar. 15, 2007

(54) Methods and therapies for potentiating therapeutic activities of a cannabinoid receptor agonist via administration of a cannabinoid receptor antagonist

(76) Inventors: Mary C. Olmstead, Kingston (CA); Jay J. Paquette, Peterborough (CA)

Correspondence Address: Kathleen A. Tyrrell;Licata & Tyrrell P.C., 66 E. Main Street, Marlton, NJ, 08053, US

(21) Appl. No.: 11/510,390

(22) Filed: Aug. 25, 2006

Related U.S. Application Data
(60) Provisional application No. 60/711,813, filed on Aug. 26, 2005.

(30) Foreign Application Priority Data
Aug. 26, 2005 (CA) 2,517,313

(51) Int. Cl. A61K 31/353; A61K 31/16

(52) U.S. Cl. 514/454; 514/625

(57) ABSTRACT

Combination therapies of a cannabinoid receptor agonist and a cannabinoid antagonist in an amount effective to potentiate, but not antagonize, the therapeutic effect of the cannabinoid receptor agonist are provided. Also provided are methods for use of these combination therapies in potentiating a therapeutic effect of cannabinoid receptor agonists, inhibiting development of acute and chronic tolerance to cannabinoid receptor agonists and treating conditions treatable with cannabinoid receptor agonists in a subject. In addition, a method for reversing cannabinoid receptor agonist tolerance and/or restoring therapeutic action of a cannabinoid receptor agonist in a subject via administration of a cannabinoid receptor antagonist in an amount effective to potentiate, but not antagonize the therapeutic effect of the cannabinoid receptor agonist is provided.

### RELATED APPLICATIONS

This patent application claims the benefit of priority from U.S. Provisional Application Ser. No. 60/711,813, filed Aug. 26, 2005, the teachings of which are herein incorporated by reference in their entirety.

### FIELD OF THE INVENTION

A combination therapy is provided for potentiating a therapeutic activity of a cannabinoid receptor agonist by co-administration with a cannabinoid receptor antagonist in an amount effective to potentiate, but not antagonize, the therapeutic activity of the cannabinoid receptor agonist. The present invention thus relates to compositions and methods for potentiating therapeutic actions of cannabinoid receptor agonists, including but not limited to, analgesia, inhibition of nausea or vomiting, treatment of glaucoma, control of muscle spasticity in movement disorders, inhibition of neurodegeneration, inhibition of anxiety, treatment of hypertension, inhibition of inflammation, treatment of Alzheimer's disease, treatment of gastrointestinal disorders such as diarrhea, and preventing or reducing atherosclerosis, and for effectively inhibiting the development of chronic as well as acute tolerance to the therapeutic actions of the cannabinoid receptor agonists via ultra-low dose cannabinoid receptor antagonist therapy. Methods for reversing cannabinoid receptor agonist tolerance and/or restoring therapeutic potency of a cannabinoid receptor agonist via administration of an ultra-low dose of a cannabinoid receptor antagonist to a subject receiving cannabinoid receptor agonist therapy are also provided.

### BACKGROUND OF THE INVENTION

Cannabinoids, like (-)-trans-delta-9-tetrahydrocannabinol (delta-9-THC) and similar natural, endogenous, and/or synthetic compounds, produce a range of behavioral and other effects (e.g., catalepsy, hypothermia, analgesia, disruption of psychomoter behavior, short term memory impairment, intoxication, stimulation of appetite, and anti-emetic effects), although tolerance to these effects may develop with repeated use (Dewey, W. L., Pharmacol. Rev. 1986 38:151-178; also see Iversen L. Brain 2003 126:1252-1270).

Control of pain via administration of the endogenous cannabinoids anandamide and palmitoylethanolamine is described in U.S. Pat. No. 6,348,498 and 6,656,972.

Cannabinoid receptor agonists are also used and/or are being investigated for use in inhibition of nausea and/or vomiting (e.g. Nabilone, Cambridge Laboratories); inhibition of neurodegeneration (Shen, M. and Thayer, S. A. Molecular Pharmacology 1998 54:459-462); inhibition of anxiety (Kathuria, S. et al., Nature Medicine 2003 9:76-81); treatment of gastrointestinal disorders such as diarrhea (Izzo et al. Naunyn Schmiedebergs Arch Pharmacol 1999 359(1):65-70); treatment of acute inflammation related to, for example, trauma and chronic inflammation related to autoimmune diseases, such as rheumatoid arthritis, Crohn's disease, ulcerative colitis (Federico M. et. al. Clin. Invest. 2004 113:1202-1209) and pulmonary inflammation (Berdyshev et al. Life Sci. 1998 63(8):PL125-9); treatment of glaucoma (Järvinen et al., Pharmacology & Therapeutics 2002 95(2):203-220); treatment of hypertension (U.S. Pat. No. 6,903,137); treatment of movement disorders/diseases such as Parkinson's disease; and treatment or prevention of atherosclerosis (Steffens et al. Nature 2005 434:782-786).

Pharmacologically, cannabinoids bind to G-protein coupled cannabinoid CB1 receptors, located primarily in neural membranes (Matsuda et al. Nature 1990 346:561-4) and to CB2 receptors found primarily in cells of the immune system, for example macrophages in the marginal zone of spleen, and peripheral neurons (Munro et al. Nature 1993 365:61-5). 2-Arachidonylglycerol, an endogenous ligand for CB1 and CB2 receptors has been suggested to induce the migration of several types of leukocytes such as macrophage/monocytes through a CB2-receptor dependent mechanism, thereby stimulating inflammatory reactions and immune response (Kishimoto et al. J. Biol. Chem. 2003 278(27):24469-24475).

CB1 receptor agonists produce dose-dependent analgesic effects, and reduce the neurotransmission in pain-related pathways (Meng et al. Nature 1998 395:381-3). Furthermore, activation of the CB1 receptor activates inhibitory Gi proteins which then down-regulate adenylyl cyclase production of cAMP (Howlett, A. C., Mol. Pharmacol. 1985 27:429-36; Howlett, A. C. and Fleming, R. M. Mol. Pharmacol. 1984 26:532-8; Howlett et al. Mol. Pharmacol. 1986 29:307-13). Previous studies suggested that Gi protein activation by CB1 receptors is necessary for cannabinoid-induced analgesia (Raffa et al. Neurosci Lett 1999 263:29-32).

More recent evidence, however, is indicative of the behavioral pharmacology of cannabinoids being more complex. For example, cannabinoid CB1 receptor agonists have been reported to have dose-dependent biphasic effects on behavior. In particular, low doses of the endocannabinoid anandamide (10 μg/kg) were demonstrated to produce increased locomotion, rearing, defecation, and nociception, and decreased catalepsy: effects that are opposite to higher doses (10 mg/kg) (Sulcova et al. Pharmacol. Biochem. Behav. 1998 59:347-52). Furthermore, the CB1 receptor has been suggested to couple to both the inhibitory Gi-protein, and the stimulatory Gs-protein (Glass, M. and Felder, C. C., J Neurosci 1997 17:5327-33; Calandra et al. Eur J Pharmacol. 1999 374:445-55). The ability of the CB1 receptor population to couple to both stimulatory and inhibitory G-proteins may explain cannabinoid-induced biphasic effects on behavior.

A synergy between low dose treatment with opioids and cannabinoid receptor antagonists at reducing the motivation to consume alcohol in rats has been disclosed (Gallate et al. Psychopharmacology 2004 173:210-216).

The opioid and cannabinoid systems have also been disclosed to be involved in the control of appetite with the cannabinoid receptor antagonist SR 141716 attenuating overfeeding induced by morphine administered systemically or intracranially into the paraventricular nucleus of the hypothalamus, but not food intake induced by administration of morphine intracranially into the nucleus accumbens (Verty et al. Psychopharmacology 2003 168:314-323). Peripheral but not central administration of the cannabinoid receptor agonist WIN 55 212-2 was reported to promote hyperphagia in partially satiated rats while peripheral, but not central administration of SR 141716 reduced food intake in rats (Gomez et al. J. Neurosci. 2002 22(21):9612-9617). The selective CB1 cannabinoid receptor antagonist SR 141716 (also referred to as ACOMPLIA or RIMONABANT) is currently under development by Sanofi-Aventis for treatment of obesity (see drugdevelopment-technology with the extension .com/projects/rimonabant/ or sanofi-synthelabo with the extension us/live/us/en, both of the world wide web).

U.S. Pat. No. 5,547,524 discloses aryl-benzo[b]thiophene and benzo[b]furan compounds which are antagonists of the CB-1 receptor in the mammalian central nervous system. These compounds are suggested to be useful in treating a variety of disorders associated with cannabinoid stimulation including depression, cognitive dysfunction, loss of memory and poor alertness and sensory perception.

Published U.S. Patent Application US2004/0209861 discloses the combination of a CB1 receptor antagonist and a compound which activates dopaminergic neurotransmission in the brain in the treatment of Parkinson's disease.

### SUMMARY OF THE INVENTION

An aspect of the present invention is a composition comprising a cannabinoid receptor agonist, in an amount effective to produce a desired therapeutic effect, and a cannabinoid receptor antagonist, in an amount effective to potentiate, but not antagonize, the therapeutic effect of the cannabinoid receptor agonist. These compositions provide useful therapeutic agents for treatment of pain, nausea or vomiting, glaucoma, a movement disorder, neurodegeneration, anxiety, acute inflammation, chronic inflammation, pulmonary inflammation, Alzheimer's disease, gastrointestinal disorders such as diarrhea, hypertension and atherosclerosis.

Another aspect of the present invention is a method for potentiating a therapeutic effect of a cannabinoid receptor agonist in a subject which comprises administering to the subject, in combination with a cannabinoid receptor agonist, a cannabinoid receptor antagonist in an amount effective to potentiate, but not antagonize the therapeutic effect of the cannabinoid receptor agonist.

Another aspect of the present invention is a method for inhibiting development of acute tolerance to a therapeutic action of a cannabinoid receptor agonist in a subject which comprises administering to the subject, in combination with a cannabinoid receptor agonist, a cannabinoid receptor antagonist in an amount effective to potentiate, but not antagonize, the therapeutic effect of the cannabinoid receptor agonist.

Another aspect of the present invention is a method for inhibiting development of chronic tolerance to a therapeutic action of a cannabinoid receptor agonist in a subject which comprises administering to the subject, in combination with a cannabinoid receptor agonist, a cannabinoid receptor antagonist in an amount effective to potentiate, but not antagonize, the therapeutic effect of the cannabinoid receptor agonist.

Another aspect of the present invention is a method for reversing tolerance to a therapeutic action of a cannabinoid receptor agonist and/or restoring therapeutic potency of a cannabinoid receptor agonist in a subject which comprises administering a cannabinoid receptor antagonist to a subject receiving a cannabinoid receptor agonist, said cannabinoid receptor antagonist being administered in an amount effective to potentiate, but not antagonize the therapeutic effect of the cannabinoid receptor agonist.

Another aspect of the present invention is a method for treating a subject suffering from a condition treatable with a cannabinoid receptor agonist comprising administering to the subject a cannabinoid receptor agonist in an amount effective to produce a therapeutic effect and a cannabinoid receptor antagonist in an amount effective to potentiate, but not antagonize, the therapeutic effect of the cannabinoid receptor agonist.

The above methods are useful for treating subjects suffering from conditions including, but not limited to, pain, nausea or vomiting, glaucoma, a movement disorder, neurodegeneration, anxiety, acute inflammation, chronic inflammation, pulmonary inflammation, Alzheimer's disease, gastrointestinal disorders such as diarrhea, hypertension and atherosclerosis. It is understood that such treatment may also be commenced prior to such suffering (i.e., prophylactically, when the subject is at risk for such suffering).

In preferred embodiments, the above methods are useful for treating post-surgical pain, pain related to multiple sclerosis, pain related to cancer, and neuropathic pain.

Yet a further aspect of the present invention in each of the above methods is that the cannabinoid receptor antagonist is administered or formulated in an amount which potentiates, but does not antagonize, the therapeutic effect of the cannabinoid receptor agonist, and that the amount of the cannabinoid receptor antagonist, alone or in combination with the cannabinoid receptor agonist, does not elicit a substantial undesirable side effect.

### BRIEF DESCRIPTION OF THE FIGURES

FIG. 1A and 1B are line graphs illustrating the antinociceptive properties of the cannabinoid receptor agonist WIN 55 212-2 (WIN) in a rat tail flick test being enhanced by an ultra-low dose of the cannabinoid CB1 receptor antagonist SR 141716 (SR) following a single injection of this combination therapy. Group mean (+/−SEM) antinociception is represented as percent mean possible effect (MPE) using the tail flick test. In FIG. 1A the cannabinoid receptor agonist WIN was administered at 0.0625 mg/kg and the cannabinoid CB1 receptor antagonist SR was administered at the ultra-low doses of 0.55 ng/kg and 0.055 ng/kg. In FIG. 1B, the cannabinoid receptor agonist WIN was administered at 0.09375 mg/kg and the cannabinoid CB1 receptor antagonist SR was administered at the ultra-low doses of 0.83 ng/kg and 0.083 ng/kg. Vehicle data are re-plotted from FIG. 1A in FIG. 1B. Animals receiving vehicle alone are depicted by filled circles. Animals receiving WIN alone are depicted by open circles. Animals receiving WIN plus SR at 0.55 ng/kg (FIG. 1A) or 0.83 ng/kg (FIG. 1B) are depicted by filled triangles. Animals receiving WIN plus SR at 0.055 ng/kg (FIG. 1A) or 0.083 ng/kg (FIG. 1B) are depicted by open triangles. Animals receiving SR alone at 0.55 ng/kg (FIG. 1A) or 0.83 ng/kg (FIG. 1B) are depicted by filled squares. Each group had 8 animals. All drugs were administered via intravenous injection. The symbol “@” is representative of a significant difference from vehicle. The symbol “*” is representative of a significant difference from WIN 0.0625 mg/kg alone. The symbol “*” is representative of a significant difference from WIN 0.09375 mg/kg alone.

FIG. 2 is a line graph illustrating that daily administrations of an ultra-low dose of the cannabinoid CB1 receptor antagonist SR 141716 (SR) with the cannabinoid receptor agonist WIN 55 212-2 (WIN) prevented the development of tolerance to the analgesic action of the cannabinoid receptor agonist in the rat tail flick test. Group mean (+/−SEM) antinociception is represented as percent mean possible effect (MPE). Each group had 8 animals. All drugs were administered via intravenous injection. Animals receiving vehicle alone are depicted by filled circles. Animals receiving WIN at 0.125 mg/kg alone are depicted by open circles. Animals receiving WIN plus SR at 1.1 ng/kg are depicted by filled triangles. Animals receiving WIN plus SR at 0.11 ng/kg are depicted by open triangles. Animals receiving SR alone at 1.1 ng/kg are depicted by filled squares. The symbol “@” is representative of a significant difference from vehicle. The symbol “*” is representative of a significant difference from WIN 0.125 mg/kg alone.

### DETAILED DESCRIPTION OF THE INVENTION

Cannabinoid CB1 receptor antagonists are being developed as a new class of therapeutic agents for drug addiction (see Le Foll, B. and Goldberg, S. R., J. Pharmacol. and Exp. Therapeutics 2005 312(3):875-883 for review). Further, it has been suggested that cannabinoid receptor antagonists may alter endogenous opioid peptide release, thus facilitating a reduction in alcohol consumption (Manzanares et al. Alcohol and Alcoholism 2005 40(1):25-34). Cannabinoid receptor agonists have been investigated for the treatment of Alzheimer's disease (Ramirez et al., The Journal of Neuroscience. 2005 25(8):1904-1913). Cannabinoids bind to CB2 receptors found primarily in cells of the immune system, for example macrophages in the marginal zone of spleen, and peripheral neurons (Stander et al. J. Dermatol. Sci. 2005 38:177-88). Published U.S. Patent Application US2005/0014786 discloses tetrahydroquinoline containing compounds which are cannabinoid-1 receptor modulators and include selective agonists, partial agonists, inverse agonists, antagonists or partial antagonists of the cannabinoid receptor. Preferred are compounds possessing activity as antagonists or inverse agonists of the CB-1 receptor taught to be useful in metabolic disorders or psychiatric disorders. U.S. Pat. No. 6,825,209 discloses amide analogs of the cannabinoid receptor antagonist SR 141716 with increased CB1 receptor selectivity which are suggested to be useful in treatment of CB1 receptor related disorders such as obesity, schizophrenia, memory dysfunction and marijuana abuse.

It has now been found that administration of an ultra-low dose of a cannabinoid receptor antagonist potentiates regional cannabinoid receptor agonist analgesia and inhibits, delays and/or reduces the development of tolerance to cannabinoid receptor agonists. It is expected that administration of an ultra-low dose of cannabinoid receptor antagonist will also reverse, decrease or inhibit tolerance to a cannabinoid receptor agonist in a subject partially or fully tolerant to cannabinoid receptor agonist therapy and/or restore, at least partially, potency of a cannabinoid receptor agonist in such a subject. The present invention provides new combination therapies for potentiating a therapeutic activity of a cannabinoid receptor agonist and inhibiting, preventing or decreasing development of chronic and/or acute tolerance to a cannabinoid receptor agonist involving co-administration of a cannabinoid receptor agonist with a cannabinoid receptor antagonist. An aspect of the present invention thus relates to compositions comprising a cannabinoid receptor agonist and an ultra-low dose of a cannabinoid receptor antagonist. Another aspect of the present invention relates to methods for potentiating a therapeutic action of a cannabinoid receptor agonist and/or effectively inhibiting or decreasing the development of acute as well as chronic tolerance to a therapeutic action of a cannabinoid receptor agonist by co-administering the cannabinoid receptor agonist with an ultra-low dose of a cannabinoid receptor antagonist. Another aspect of the present invention relates to methods for reversing tolerance to a therapeutic action of a cannabinoid receptor agonist and/or restoring therapeutic potency of a cannabinoid receptor agonist by administering an ultra-low dose of a cannabinoid receptor antagonist to a subject already receiving a cannabinoid receptor agonist. The new combination therapies of the present invention are expected to be useful in optimizing the use of cannabinoid drugs in various applications including but not limited to: pain management, e.g. management of acute post-surgical pain, obstetrical pain, acute or chronic inflammatory pain, pain associated with conditions such as multiple sclerosis or cancer, pain associated with trauma, pain associated with migraines, neuropathic pain, and central pain; management of chronic pain syndrome of a non-malignant origin such as chronic back pain; inhibition of nausea and/or vomiting; treatment of glaucoma; and inhibiting spasticity and controlling movement in movement disorders such as Parkinson's disease.

Cannabinoid receptor antagonists useful in the combination therapies and methods of the present invention include any compound that partially or completely reduces, inhibits, blocks, inactivates and/or antagonizes the binding of a cannabinoid receptor agonist to its receptor to any degree and/or the activation of a cannabinoid receptor to any degree. Thus, the term cannabinoid receptor antagonist is also meant to include compounds that antagonize the agonist in a competitive, irreversible, pseudo-irreversible and/or allosteric mechanism. In addition, the term cannabinoid receptor antagonist includes compounds at a low dose or ultra-low dose that increase, potentiate and/or enhance the therapeutic and/or analgesic potency and/or efficacy of cannabinoid receptor agonists, while at similar doses does not demonstrate a substantial or significant antagonism of a cannabinoid receptor agonist. Cannabinoid receptor antagonists useful in the combination therapies and methods of the present invention include, but are in no way limited to, antagonists of cannabinoid 1 (CB1) receptors, antagonists of cannabinoid 2 (CB2)receptors, and antagonists of both CB1 and CB2 receptors. In a preferred embodiment, the cannabinoid receptor antagonist is a CB1 receptor antagonist. Examples of cannabinoid receptor antagonists useful in the present invention include, but are in no way limited to, SR 141716, AM-251 (Tocris Cookson, Bristol, UK), AM281 (Tocris Cookson, Bristol, UK) LY320135 (Eli Lilly, Inc. Indiana), and SR 144528 (Rinaldi-Carmona et al. J Pharmacol Exp Ther 1998 284:644-650). Exemplary cannabinoid receptor antagonists useful in the present invention are also set forth in U.S. Pat. No. 6,825,209, 5,547,524, and 6,916,838 and published U.S. Patent Application 2005/0014786. In some embodiments, preferred cannabinoid receptor antagonists are SR 141716 and LY320135.

Compositions of the present invention as well as methods described herein for their use may comprise an ultra-low dose of more than one cannabinoid receptor antagonist alone, or more than one cannabinoid receptor antagonist at an ultra-low dose in combination with one or more cannabinoid receptor agonist.

The cannabinoid receptor antagonist is included in the compositions and administered in the methods of the present invention at an ultra-low dose. By ultra-low dose, as used herein, it is meant an amount of cannabinoid receptor antagonist that potentiates, but does not antagonize, a therapeutic effect of a cannabinoid receptor agonist. Thus, in one embodiment, by the term “ultra-low dose” it is meant an amount of the cannabinoid receptor antagonist lower than that established by those skilled in the art to significantly block or inhibit cannabinoid receptor activity.

As used herein, the term “amount” is intended to refer to the quantity of cannabinoid receptor antagonist and/or agonist administered to a subject. The term “amount” encompasses the term “dose” or “dosage”, which is intended to refer to the quantity of cannabinoid receptor antagonist and/or agonist administered to a subject at one time or in a physically discrete unit, such as, for example, in a pill, injection, or patch. The term “amount” also encompasses the quantity of cannabinoid receptor antagonist and/or agonist administered to a subject, expressed as the number of molecules, moles, grams, or volume per unit body mass of the subject, such as, for example, mol/kg, mg/kg, ng/kg, ml/kg, or the like, sometimes referred to as concentration administered.

In accordance with the invention, administration to a subject of a given amount of cannabinoid receptor antagonist and/or agonist results in an effective concentration of the antagonist and/or agonist in the subject's body. As used herein, the term “effective concentration” is intended to refer to the concentration of cannabinoid receptor antagonist and/or agonist in the subject's body (e.g., in the blood, plasma, or serum, at the target tissue(s), or site(s) of action) capable of producing a desired therapeutic effect. The effective concentration of cannabinoid receptor antagonist and/or agonist in the subject's body may vary among subjects and may fluctuate within a subject over time, depending on factors such as, but not limited to, the condition being treated, genetic profile, metabolic rate, biotransformation capacity, frequency of administration, formulation administered, elimination rate, and rate and/or degree of absorption from the route/site of administration. For at least these reasons, for the purpose of this disclosure, administration of cannabinoid receptor antagonist and/or agonist is conveniently provided as amount or dose of cannabinoid receptor antagonist or agonist. The amounts, dosages, and dose ratios provided herein are exemplary and may be adjusted, using routine procedures such as dose titration, to provide an effective concentration.

In one embodiment the amount of cannabinoid receptor antagonist administered potentiates, but does not antagonize, a therapeutic effect of a cannabinoid receptor agonist. Thus, the effective concentration of a cannabinoid receptor antagonist is a concentration in the body which potentiates the therapeutic action of a cannabinoid receptor agonist. Preferably, the amount of cannabinoid receptor antagonist administered potentiates the therapeutic action of the cannabinoid receptor agonist without the amount of the cannabinoid receptor antagonist, alone or in combination with the cannabinoid receptor agonist, eliciting a substantial undesirable effect.

For example, in one embodiment, an ultra-low dose of cannabinoid receptor antagonist is an amount ineffective at G_{i/o}-coupled CB1 receptor blockade. For example, an ultra-low dose of SR 141716 demonstrated herein to potentiate the analgesic activity of the cannabinoid receptor agonist WIN 55 212-2 is approximately 300,000-fold lower than an optimal dose of SR 141716 producing a blockade of the G_{i/o}-coupled CB1 receptors. An ultra-low dose useful in the present invention for other cannabinoid CB1 receptor antagonists can be determined routinely by those skilled in the art in accordance with their known doses as G_{i/o}-coupled CB1 receptor blockers and the methodologies described herein for SR 141716. In general, in this embodiment, however, by “ultra-low” it is meant a dose of cannabinoid receptor antagonist of at least 1,000- to 100,000,000-fold lower than a dose of cannabinoid receptor antagonist producing a blockade of G_{i/o}-coupled CB1 receptors. As will be understood by the skilled artisan upon reading this disclosure, however, other means for measuring cannabinoid receptor antagonism can be used.

An exemplary embodiment of an “ultra-low dose” is an amount of cannabinoid receptor antagonist which is significantly less than the amount of cannabinoid receptor agonist to be administered. Thus, in this embodiment, the ultra-low dose of cannabinoid receptor antagonist is expressed as a ratio with respect to the dose of cannabinoid receptor agonist administered, or to be administered. In this embodiment a preferred ratio for an ultra-low dose is a ratio of 1:10,000, 1:100,000, 1:1,000,000 or 1:100,000,000 or any ratio in between of cannabinoid receptor antagonist to cannabinoid receptor agonist.

In another embodiment, the cannabinoid receptor antagonist and cannabinoid receptor agonist are administered to a subject in amounts that result in relative ratios of amounts or effective concentrations within the blood, plasma, serum, or at the target tissue(s), or site(s) of action of 1:10,000, 1:100,000, 1:1,000,000 or 1:100,000,000 or any ratio in between.

Another exemplary embodiment of an “ultra-low” dose is an amount or ratio which potentiates the therapeutic action of the cannabinoid receptor agonist without the amount of the cannabinoid receptor antagonist, alone or in combination with the cannabinoid receptor agonist, eliciting a substantial undesirable effect.

By “substantial undesirable side effect” as used herein it is meant a response in a subject to the cannabinoid receptor antagonist other than potentiating the therapeutic action of the cannabinoid receptor agonist which can not be controlled in the subject and/or endured by the subject and/or could result in discontinued treatment of the subject with the combination therapies and methods of the present invention.

Examples of such side effects include tolerance, dependence, addiction, sedation, euphoria, dysphoria, memory impairment, hallucination, depression, dry mouth, muscle weakness, slurred speech, increased heart rate, decrease in blood pressure, dizziness, and headache.

Cannabinoid receptor agonists useful in the combination therapies and methods of the present invention include any compound (either endogenous or exogenous to the subject) that binds to and/or activates and/or agonizes a cannabinoid receptor to any degree and/or stabilizes the cannabinoid receptor in an active or inactive conformation. Thus, by the term cannabinoid receptor agonist it is meant to include partial agonists, inverse agonists, as well as complete agonists of a cannabinoid receptor. By cannabinoid receptor agonist it is also meant to be inclusive of compounds that enhance the activity of cannabinoid receptor agonist compounds produced within the body (i.e., endocannabinoids), as well as exogenous cannabinoid receptor agonists (i.e., synthetic ornaturally-occurring). For example, anandamide transporter inhibitors block the uptake of endogenously produced and released anandamide (an endocannabinoid) and thus leave more anandamide in the synapse to activate the cannabinoid receptor. Similarly, inhibitors of enzymes such as fatty acid amide hydrolase (FAAH), which metabolizes anandamide, can prevent cannabinoid receptor agonist metabolism, thereby leaving more anandamide available to activate the cannabinoid receptor. Accordingly, for purposes of the present invention, by cannabinoid receptor agonist it is meant to be inclusive of endocannabinoid transporter inhibitors and cannabinoid metabolizing enzyme inhibitors.

Examples of cannabinoid receptor agonists useful in the combination therapies and methods of the present invention include, but are in no way limited to endocannabinoid, (-)-trans-delta-9-tetrahydrocannabinol (delta-9-THC), CP-55,940, arachidonylethanolamide (anandamide), WIN 55 212-2, HU-210, HU-243, arachidonyl-2-chloroethylamide, arachidonylcyclopropylamide, O-1812, 2-arachidonoyl glycerol, dronabinol (marinol), sativex, cannabidiol, cannabinol, cannabichromene, cannabigerol and phytocannabinoids, endocannabinoid transporter inhibitors including, but not limited to, AM404, VDM, UCM 707, OMDM-2, LY 2183240 and (−)-5′-DMH-CDB and endocannabinoid metabolizing enzyme inhibitors including, but not limited to, palmitoylisopropylamide, 1,1-trifluro-6Z,9Z,12Z,15Z-heneicosateraen-2-one or arachidonyl trifluoromethyl ketone (AACOCF₃) and 5Z, 8Z, 11Z, 14Z)-5,8,11,14-eicosatetraenyl-methyl ester phosphonofluoridic acid (MAFP). In some embodiments, preferred cannabinoid receptor agonists are delta-9-THC, WIN 55 212-2, cannabidiol, and AM404.

Compositions of the present invention as well as methods described herein for their use may comprise more than one cannabinoid receptor agonist and/or more than one cannabinoid receptor antagonist, formulated and/or administered in various combinations.

Preferred combinations of cannabinoid receptor agonists and cannabinoid receptor antagonists used in the present invention include WIN 55 212-2/SR141716, which is demonstrated herein to be effective in animal models. Another preferred combination is THC/SR141716 as both of these drugs are effective in humans. Another preferred combination is ACEA/AM251 as these drugs target the CB1 receptor. Yet another preferred combination is an endocannabinoid transporter inhibitor such as AM404, which exhibits CB1 agonistic properties by increasing the levels of the endocannabinoid anandamide in the synaptic space, and SR141716.

The dose of cannabinoid receptor agonist included in the compositions of the present invention and used in the methodologies described herein is an amount that achieves an effective concentration and/or produces a desired therapeutic effect. For example, such a dosage may be an amount of cannabinoid receptor agonist well known to the skilled artisan as having a therapeutic action or effect in a subject. Dosages of cannabinoid receptor agonist producing, for example, an analgesic effect can typically range between about 0.02 mg/kg to 100 mg/kg, depending upon, but not limited to, the cannabinoid receptor agonist selected, the route of administration, the frequency of administration, the formulation administered, and/or the condition being treated. Further, as demonstrated herein, co-administration of a cannabinoid receptor agonist with an ultra-low dose of a cannabinoid receptor antagonist potentiates the analgesic effect of the cannabinoid receptor agonist. Thus, when co-administered with a cannabinoid receptor antagonist, the amount or dose of cannabinoid receptor agonist effective at producing a therapeutic effect may be lower than when the cannabinoid receptor agonist is administered alone.

For purposes of the present invention, by “therapeutic effect” or “therapeutic activity” or “therapeutic action” it is meant a desired pharmacological activity of a cannabinoid receptor agonist useful in the inhibition, prevention, mitigation, reduction or treatment of pain, nausea or vomiting, glaucoma, a movement disorder, neurodegeneration, anxiety, acute inflammation, chronic inflammation, pulmonary inflammation, Alzheimer's disease, a gastrointestinal disorder such as diarrhea, hypertension or atherosclerosis.

For purposes of the present invention, by potentiate, it is meant that administration of the cannabinoid receptor antagonist enhances, extends or increases, at least partially, the therapeutic activity of the cannabinoid receptor agonist and/or results in a decreased amount of cannabinoid receptor agonist being required to produce a desired therapeutic effect. Thus, as will be understood by the skilled artisan upon reading this disclosure, the amount of cannabinoid receptor agonist included in the combination therapy of the present invention may be decreased as compared to an established amount of the cannabinoid receptor agonist when administered alone. The amount of the decrease for other cannabinoid receptor agonists can be determined routinely by the skilled artisan based upon ratios described herein for WIN 55 212-2 and SR 141716.

This decrease in required amount of cannabinoid receptor agonist to achieve the same or similar therapeutic benefit may decrease any unwanted side effects associated with cannabinoid receptor agonist therapy. Thus, the combination therapies of the present invention also provide a means for decreasing unwanted side effects of cannabinoid receptor agonist therapy alone.

By “antagonize” as used herein, it is meant an inhibition or decrease in therapeutic effect or action of a cannabinoid receptor agonist resulting from addition of a cannabinoid receptor antagonist which renders the cannabinoid receptor agonist ineffective therapeutically for the condition being treated.

By “tolerance” as used herein, it is meant a loss of drug potency and is produced by most, if not all currently used cannabinoid receptor agonists. Chronic or acute tolerance can be a limiting factor in the clinical management of cannabinoid receptor agonist drugs as cannabinoid receptor agonist potency is decreased upon exposure to the cannabinoid receptor agonist. By “chronic tolerance” as used herein, it is meant a decrease in potency which can develop after drug exposure over several or more days. However, loss of cannabinoid receptor agonist drug potency may also be seen in pain conditions such as neuropathic pain without prior cannabinoid receptor agonist drug exposure as neurobiological mechanisms underlying the genesis of tolerance and neuropathic pain are similar (Mao et al. Pain 1995 61:353-364) This type of tolerance is referred to herein as “acute tolerance”.

Tolerance has been explained in terms of drug receptor desensitization or internalization. It has also been explained on the basis of an adaptive increase in levels of pain transmitters such as glutamic substance P or CGRP, and in the case of the opioid system a switch in opioid receptor coupling from Gαi/o to Gαs associated G proteins. Inhibition of tolerance and maintenance of cannabinoid receptor agonist potency are important therapeutic goals in cannabinoid receptor agonist therapies which, as demonstrated herein, are achieved via the combination therapies of the present invention.

The ability of the combination therapies of the present invention to potentiate the therapeutic action of analgesia of a cannabinoid receptor agonist and/or inhibit chronic cannabinoid receptor agonist tolerance upon co-administration of an ultra-low dose of a cannabinoid receptor antagonist was demonstrated in tests of thermal (rat tail flick) antinociception. In these experiments, the cannabinoid CB1 receptor antagonist used was SR 141716 (referred to herein as SR). The cannabinoid receptor agonist was WIN 55 212-2 (referred to herein as WIN). As will be understood by the skilled artisan upon reading this disclosure, however, the combination of cannabinoid receptor agonist and cannabinoid receptor antagonist selected for these experiments as well as the therapeutic action measured are merely exemplary and are in no way limiting to the scope of this invention.

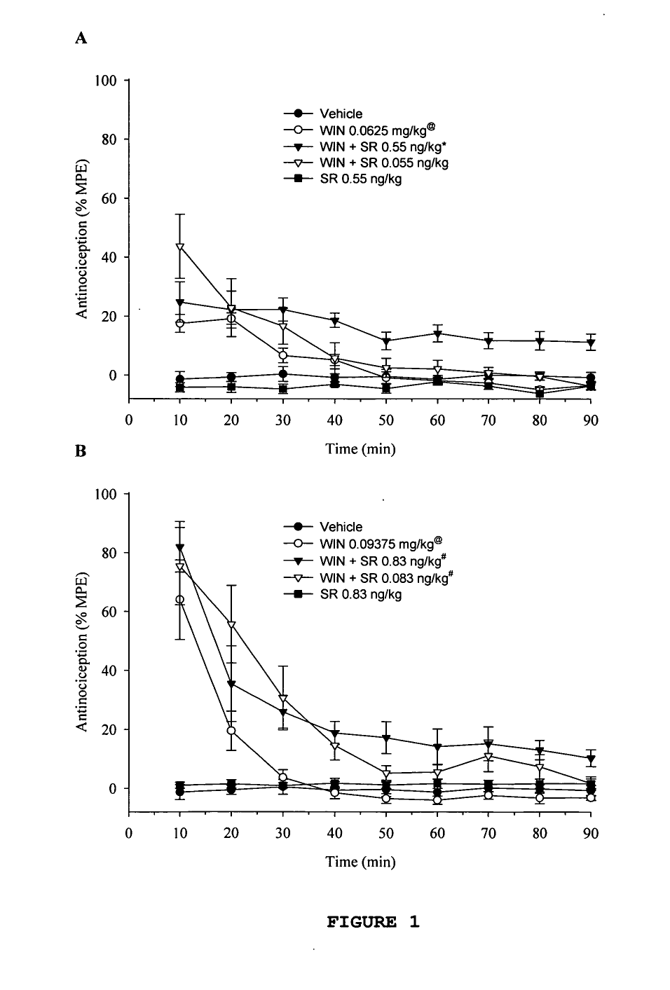

FIGS. 1A and 1B show the tail flick data from animals given single injections of the cannabinoid receptor agonist WIN alone, the cannabinoid receptor antagonist SR alone, or a combination of the cannabinoid receptor agonist WIN and the cannabinoid receptor antagonist SR. In FIG. 1A animals administered a cannabinoid receptor agonist received 0.625 mg/kg WIN. Animals receiving a cannabinoid CB1 receptor antagonist were administered either 0.55 ng/kg or 0.055 ng/kg SR. In FIG. 1B animals administered a cannabinoid receptor agonist received 0.09375 mg/kg WIN. Animals receiving a cannabinoid receptor antagonist were administered either 0.83 ng/kg or 0.083 ng/kg SR. Nociceptive testing was performed every 10 minutes following administration of the therapeutic compounds over a 90 minute period. When all drug groups were combined, a main effect of time [F(3.51,221.11)=66.64, P<0.001] was revealed, whereby tail flick latency mean possible effects (MPEs) decreased across time. Furthermore, there was a main drug effect [F(8,63)=16.00, P<0.001], and a drug X post-injection time interaction, [F(28.08,221.11)=7.62, P<0.001].

When comparing drug groups, there was no difference between vehicle and 0.0625 mg/kg WIN-treated animals [t(14)=2.63], but tail flick latencies of vehicle-treated animals were significantly different from those of animals injected with 0.09375 WIN [t(14)=4.629, P≦0.005]. The tail flick latency MPEs were also compared between WIN alone groups and the combined WIN and ultra-low dose SR groups. There was no difference between the 0.0625 mg/kg WIN alone group and the same dose of WIN mixed with the 0.055 ng/kg SR [t(14)=3.28], but a slightly higher ultra-low dose of SR (0.55 ng/kg) in combination with 0.0625 mg/kg WIN showed longer tail flick latencies compared to the same dose of WIN alone [t(14)=6.72, P≦0.005]. The combination treatment of 0.09375 mg/kg WIN with both the 0.83 and 0.083 ng/kg ultra-low dose SR produced longer tail flick latencies compared to the same doses of WIN alone [t(14)=9.63, P≦0.005, and t(14)=8.16, P≦0.005 respectively]. Thus, as demonstrated by these experiments, an ultra-low dose of a cannabinoid receptor antagonist increases cannabinoid receptor agonist-induced tail flick thresholds.

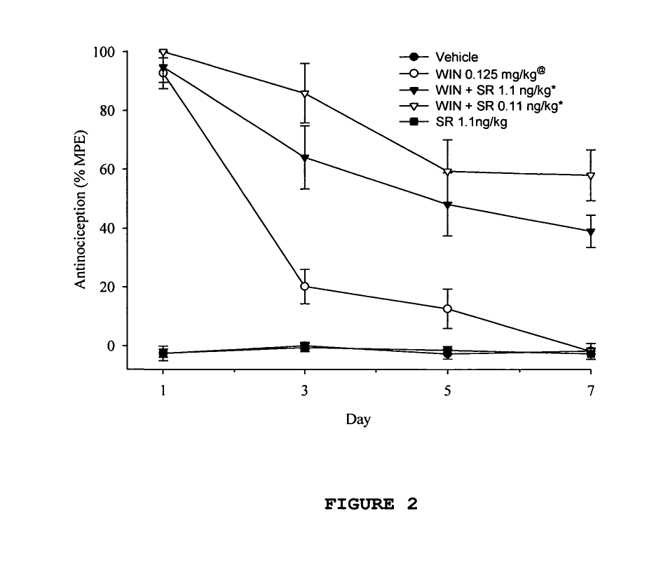

FIG. 2 shows the tail flick data from animals given repeated daily injections of a cannabinoid receptor agonist and/or a cannabinoid receptor antagonist for 7 days, and tested every other day for nociceptive responses. In this experiment, animals administered the cannabinoid receptor agonist received 0.125 mg/kg WIN. Animals administered the cannabinoid receptor antagonist received either 1.1 ng/kg or 0.11 ng/kg SR. A main effect observed in this experiment was a change in tail flick latencies across test days [F(2.98,104.30)=59.36, P<0.001]. Furthermore, there was a main effect of drug [F(4,35)=73.05, P<0.001], and a day x drug group interaction [F(11.92,104.30)=13.92, P<0.001]

When comparing drug groups, 0.125 mg/kg WIN-treated animals were significantly different from vehicle treated animals [t(14)=10.44, P≦0.01] whereby the WIN group had greater tail flick latencies. Most importantly, animals injected with 0.125 mg/kg WIN in combination with ultra-low dose SR at 1.1 and 0.11 ng/kg showed greater tail flick latency thresholds compared to WIN alone [t(14)=9.77, P≦0.01; and t(14)=14.35, P≦0.01; respectively]. Thus, as demonstrated by these experiments, an ultra-low dose of a cannabinoid antagonist blocked the development of tolerance to the antinociceptive effect of a cannabinoid receptor agonist.

On the day following the last injection, animals were sacrificed and tissue samples of the brain and lumbar spinal cord were collected. Co-immunoprecipitation experiments were performed on the tissues to determine the G-protein sub-type coupling profile of activated CB1 receptors. Results for striatal tissue indicated that WIN-induced tolerance is associated with a switch in the CB1 receptor G-protein coupling from the Gαi type to the Gαs type. Furthermore, ultra-low dose SR 141716 prevented this coupling switch. Thus, while not wishing to be bound to any particular theory, this prevention of the coupling switch by an ultra-low dose SR 141716 may be responsible for preventing WIN 55 212-2 induced antinociception.

Accordingly, as shown herein, an ultra-low dose of a cannabinoid receptor antagonist enhanced antinociception induced by a cannabinoid receptor agonist and increased the duration of the antinociceptive effect of the cannabinoid receptor agonist. Furthermore, cannabinoid receptor agonist-induced tolerance was prevented when an ultra-low dose of a cannabinoid antagonist was co-administered. Although both the SR to WIN dose ratios of 1:100,000 and 1:1,000,000 were effective, the 1:1,000,000 dose ratio appears slightly (although not significant) better at preventing WIN-induced tolerance. Thus, as shown herein, a behavioral effect of a cannabinoid receptor agonist is enhanced by a cannabinoid receptor antagonist. Further, it is believed that an ultra-low dose of a cannabinoid receptor antagonist will prevent development of acute tolerance to cannabinoid receptor agonists as well as reverse tolerance and/or restore the potency of cannabinoid receptor agonists in animals already tolerant to the analgesic action of the cannabinoid receptor agonist.

In addition to analgesia, based upon these experiments, it is expected that the combination therapies of the present invention will by useful in potentiating other therapeutic activities of cannabinoid receptor agonists, including but not limited to, inhibition of nausea or vomiting, alleviation of symptoms an/or treatment of glaucoma, and control of muscle spasticity in movement disorders.

As demonstrated herein, cannabinoid receptor agonists and cannabinoid receptor antagonists can be administered intravenously. Further, it is expected that these therapeutic compounds will be effective following other modes of systemic as well as local administration. Accordingly, the combination therapies of the invention may be administered systemically or locally, and by any suitable route such as oral, buccal, sublingual, transdermal, inhalation, subcutaneous, intraocular, intravenous, intramuscular, intrathecally, epidurally or intraperitoneal administration, and the like (e.g., by injection). Preferably, the cannabinoid receptor agonist and cannabinoid receptor antagonist are administered simultaneously via the same route of administration. However, it is expected that administration of the compounds separately, via the same route or different route of administration, within a time frame during which each therapeutic compound remains active, will also be effective therapeutically as well as in alleviating tolerance to the cannabinoid receptor agonist. Further, it is expected that administration of a cannabinoid receptor antagonist to a subject already receiving cannabinoid receptor agonist treatment will reverse any tolerance to the cannabinoid receptor agonist and restore therapeutic potency of the cannabinoid receptor agonist. Thus, treatment with the cannabinoid receptor agonist and cannabinoid receptor antagonist in the combination therapy of the present invention need not begin at the same time. For example, administration of the cannabinoid receptor antagonist may begin several days, weeks, months or more before or after treatment with the cannabinoid receptor agonist.

Accordingly, for purposes of the present invention, the therapeutic compounds, namely the cannabinoid receptor agonist and the cannabinoid receptor antagonist, can be administered together in a single pharmaceutically acceptable vehicle or separately, each in their own pharmaceutically acceptable vehicle.

As used herein, by the term “therapeutic compound”, it is meant to refer to a cannabinoid receptor agonist and/or a cannabinoid receptor antagonist.

As used herein “pharmaceutically acceptable vehicle” includes any and all solvents, excipients, dispersion media, coatings, antibacterial and antifungal agents, isotonic and absorption delaying agents, and the like which are compatible with the activity of the therapeutic compound and are physiologically acceptable to a subject. An example of the pharmaceutically acceptable vehicle is buffered normal saline (0.15 M NaCl). The use of such media and agents for pharmaceutically active substances is well known in the art. Except insofar as any conventional media or agent is incompatible with the therapeutic compound, use thereof in the compositions suitable for pharmaceutical administration is contemplated. Supplementary active compounds can also be incorporated into the compositions.

Carrier or substituent moieties useful in the present invention may also include moieties which allow the therapeutic compound to be selectively delivered to a target organ. For example, delivery of the therapeutic compound to the brain may be enhanced by a carrier moiety using either active or passive transport (a “targeting moiety”). Illustratively, the carrier molecule may be a redox moiety, as described in, for example, U.S. Pat. Nos. 4,540,654 and 5,389,623, both to Bodor. These patents disclose drugs linked to dihydropyridine moieties which can enter the brain, where they are oxidized to a charged pyridinium species which is trapped in the brain. Thus drugs linked to these moieties accumulate in the brain. Other carrier moieties include compounds, such as amino acids or thyroxine, which can be passively or actively transported in vivo. Such a carrier moiety can be metabolically removed in vivo, or can remain intact as part of an active compound. Structural mimics of amino acids (and other actively transported moieties) including peptidomimetics, are also useful in the invention. As used herein, the term “peptidomimetic” is intended to include peptide analogues which serve as appropriate substitutes for peptides in interactions with, for example, receptors and enzymes. The peptidomimetic must possess not only affinity, but also efficacy and substrate function. That is, a peptidomimetic exhibits functions of a peptide, without restriction of structure to amino acid constituents. Peptidomimetics, methods for their preparation and use are described in Morgan et al. (1989), the contents of which are incorporated herein by reference. Many targeting moieties are known, and include, for example, asialoglycoproteins (see e.g., Wu, U.S. Pat. No. 5,166,320) and other ligands which are transported into cells via receptor-mediated endocytosis (see below for further examples of targeting moieties which may be covalently or non-covalently bound to a target molecule).

The term “subject” as used herein is intended to include living organisms in which treatment with cannabinoid receptor agonists can occur. Examples of subjects include mammals such as humans, apes, monkeys, cows, sheep, goats, dogs, cats, mice, rats, and transgenic species thereof. As would be apparent to a person of skill in the art, the animal subjects employed in the working examples set forth below are reasonable models for human subjects with respect to the tissues and biochemical pathways in question, and consequently the methods, therapeutic compounds and pharmaceutical compositions directed to same. As evidenced by Mordenti (1986) and similar articles, dosage forms for animals such as, for example, rats can be and are widely used directly to establish dosage levels in therapeutic applications in higher mammals, including humans. In particular, the biochemical cascade initiated by many physiological processes and conditions is generally accepted to be identical in mammalian species (see, e.g., Mattson and Scheff, 1994; Higashi et al., 1995). In light of this, pharmacological agents that are efficacious in animal models such as those described herein are believed to be predictive of clinical efficacy in humans, after appropriate adjustment of dosage.

Depending on the route of administration, the therapeutic compound hay be coated in a material to protect the compound from the action of acids, enzymes and other natural conditions which may inactivate the compound. Insofar as the invention provides a combination therapy in which two therapeutic compounds are administered, each of the two compounds may be administered by the same route or by a different route. Also, the compounds may be administered either at the same time (i.e., simultaneously) or each at different times. In some treatment regimes it may be beneficial to administer one of the compounds more or less frequently than the other.

The compounds of the invention can be formulated to ensure proper distribution in vivo. For example, the blood-brain barrier (BBB) excludes many highly hydrophilic compounds. To ensure that the therapeutic compounds of the invention cross the BBB, they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs (“targeting moieties”), thus providing targeted drug delivery (see, e.g., Ranade et al., 1989). Exemplary targeting moieties include folate and biotin (see, e.g., U.S. Pat. No. 5,416,016 to Low et al.); mannosides (Umezawa et al., 1988); antibodies (Bloeman et al., 1995; Owais et al., 1995); and surfactant protein A receptor (Briscoe et al., 1995). In a preferred embodiment, the therapeutic compounds of the invention are formulated in liposomes; in a more preferred embodiment, the liposomes include a targeting moiety.

Delivery and in vivo distribution can also be affected by alteration of an anionic group of compounds of the invention. For example, anionic groups such as phosphonate or carboxylate can be esterified to provide compounds with desirable pharmacokinetic, pharmacodynamic, biodistributive, or other properties.

To administer a therapeutic compound by other than parenteral administration, it may be necessary to coat the compound with, or co-administer the compound with, a material to prevent its inactivation. For example, the therapeutic compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., 1984).

The therapeutic compound may also be administered parenterally (e.g., intramuscularly, intravenously, intraperitoneally, subcutaneously, intraspinally, intrathecally, intracerebrally, intraocularly, sublingually, buccally, intranasally or via inhalation). Dispersions can be prepared in glycerol, liquid polyethylene glycols, and mixtures thereof and in oils. Under ordinary conditions of storage and use, these preparations may contain a preservative to prevent the growth of microorganisms. Pharmaceutical compositions suitable for injectable use include sterile aqueous solutions (where water soluble) or dispersions and sterile powders for the extemporaneous preparation of sterile injectable solutions or dispersions. In all cases, the composition must be sterile and must be fluid to the extent that easy syringability exists. It must be stable under the conditions of manufacture and storage and must be preserved against the contaminating action of microorganisms such as bacteria and fungi. The vehicle can be a solvent or dispersion medium containing, for example, water, ethanol, polyol (for example, glycerol, propylene glycol, liquid polyethylene glycol, and the like), suitable mixtures thereof, and oils (e.g., vegetable oil). The proper fluidity can be maintained, for example, by the use of a coating such as lecithin, by the maintenance of the required particle size in the case of dispersion, and by the use of surfactants.

Prevention of the action of microorganisms can be achieved by various antibacterial and antifungal agents, for example, parabens, chlorobutanol, phenol, ascorbic acid, thimerosal, and the like. In some cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filter sterilization. Generally, dispersions are prepared by incorporating the therapeutic compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yield a powder of the active ingredient (i.e., the therapeutic compound) optionally plus any additional desired ingredient from a previously sterile-filtered solution thereof.

Solid dosage forms for oral administration include ingestible capsules, tablets, pills, lollipops, powders, granules, elixirs, suspensions, syrups, wafers, buccal tablets, troches, and the like. In such solid dosage forms the active compound is mixed with at least one inert, pharmaceutically acceptable excipient or diluent or assimilable edible carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof, or incorporated directly into the subject's diet. In the case, of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like. The percentage of the therapeutic compound in the compositions and preparations may, of course, be varied. The amount of the therapeutic compound in such therapeutically useful compositions is such that a suitable dosage will be obtained.

The solid dosage forms of tablets, dragees, capsules, pills, and granules can be prepared with coatings and shells such as enteric coatings and other coatings well-known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, ground nut corn, germ olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Therapeutic compounds can be administered in time-release or depot form, to obtain sustained release of the therapeutic compounds over time. The therapeutic compounds of the invention can also be administered transdermally (e.g., by providing the therapeutic compound, with a suitable carrier, in patch form).

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of neurological conditions in subjects.

Therapeutic compounds according to the invention are administered at a therapeutically effective dosage sufficient to achieve the desired therapeutic effect of the cannabinoid receptor agonist, e.g. to mitigate pain and/or to effect analgesia in a subject, to inhibit nausea and/or vomiting in a subject, to control and/or inhibit spasticity or a movement disorder or to alleviate a symptom of glaucoma. For example, if the desired therapeutic effect is analgesia, the “therapeutically effective dosage” mitigates pain by about 40%, preferably by about 60%, even more preferably by about 80%, and still more preferably by about 100% relative to untreated subjects. Actual dosage levels of active ingredients in the pharmaceutical compositions of this invention may be varied so as to obtain an amount of the active compound(s) that is effective to achieve the desired therapeutic response for a particular subject, compositions, and mode of administration. The selected dosage level will depend upon the activity of the particular compound, the route of administration, the severity of the condition being treated, the condition and prior medical history of the subject being treated, the age, sex, and weight of the subject, and the ability of the therapeutic compound to produce the desired therapeutic effect in the subject. Dosage regimens can be adjusted to provide the optimum therapeutic response. For example, several divided doses may be administered daily or the dose may be proportionally reduced as indicated by the exigencies of the therapeutic situation.

However, it is well known within the medical art to determine the proper dose for a particular subject by the dose titration method. In this method, the patient is started with a dose of the drug compound at a level lower than that required to achieve the desired therapeutic effect. The dose is then gradually increased until the desired effect is achieved. Starting dosage levels for an already commercially available therapeutic agent of the classes discussed above can be derived from the information already available on the dosages employed. Also, dosages are routinely determined through preclinical ADME toxicology studies and subsequent clinical trials as required by the FDA or equivalent agency. The ability of a cannabinoid receptor agonist to produce the desired therapeutic effect may be demonstrated in various well known models for the various conditions treated with these therapeutic compounds. For example, mitigation of pain can be evaluated in model systems that may be predictive of efficacy in mitigating pain in human diseases and trauma, such as animal model systems known in the art (including, e.g., the models described herein).

Compounds of the invention may be formulated in such a way as to reduce the potential for abuse of the compound. For example, a compound may be combined with one or more other agents that prevent or complicate separation of the compound therefrom.

The following nonlimiting examples are provided to further illustrate the present invention. The contents of all references, pending patent applications, and published patents cited throughout this application are hereby expressly incorporated by reference.

### EXAMPLES

### Example 1

### Animals

Male Long-Evans rats (N=175) from Charles River (Montreal, QC, Canada) ranging from 230-380 grams, were housed in polycarbonate cages in pairs and given free access to food (Lab Diet, PMI Nutrition International, Inc., Brentwood, Mo., USA) and water. Animal quarters were kept on a reverse light-dark cycle (lights on from 7 pm to 7 am) and maintained at 22±2° C. and 45±20% relative humidity. Animals were given a minimum of 3 days prior to the experiment to acclimatize to the animal quarters.

### Example 2

### Drugs and Administration

All injections were administered in a volume of 1 ml/kg. All chemicals were dissolved in 5% dimethyl sulfoxide (DMSO; Sigma, Oakville, ON, Canada), 0.3% polyoxyethylenesorbitan monooleate (Tween® 80; Sigma, Oakville, ON, Canada) and 94.7% saline vehicle, and administered intravenously in the posterior ⅓ of the lateral tail vein.

For single injection testing, vehicle alone was used as a control injection (n=8). The non-specific CB receptor agonist WIN 55 212-2 [(R)-(+)-[2,3-Dihydro-5-methyl-3-(4-morpholinylmethyl)pyrrolo[1,2,3-de]-1,4-benzoxazin-6-yl]-1-naphthalenylmethanone mesylate (WIN); Tocris Cookson, Ellisville, Mo., USA), was administered alone at doses of 0.0625 and 0.09375 mg/kg. These doses were chosen because they were shown to produce sub-maximal antinociception in the tail flick test.

The CB1 receptor antagonist SR 141716 (SR) [N-(Piperidin-1-yl)-5-(4-chlorophenyl)-1-(2,4-dichlorophenyl)-4-methyl-1H-pyrazole-3-carboxamide] was obtained from the National Institute of Mental Health's Chemical Synthesis and Drug Supply Program (Bethesda, Md., USA). An ultra-low dose of SR (0.55 or 0.055 ng/kg) was combined with WIN (0.0625 mg/kg) and administered as a single injection. These combinations produce WIN to SR molar ratios of 100,000:1 and 1,000,000:1, respectively. Also, WIN (0.09375 mg/kg) was combined with ultra-low doses of SR (0.83 or 0.083 ng/kg) producing WIN to SR molar ratios of 100,000:1 and 1,000,000:1, respectively. The control group received 0.55 and 0.83 ng/kg ultra-low dose of SR alone.

For repeated injection testing, vehicle alone was used as a control injection (n=8). WIN 55 212-2 was administered alone at doses of 0.125 mg/kg. This dose was chosen because it produces maximal antinociception in the tail flick test. Ultra-low doses of SR (1.1 or 0.11 ng/kg) were combined with WIN (0.125 mg/kg) and each time administered as a single injection. These combinations produce WIN to SR molar ratios of 100,000:1 and 1,000,000:1 respectively. The control group received 1.1 ng/kg ultra-low dose of SR alone.

### Example 3

### Apparatus

The tail flick apparatus consisted of a projection lamp that creates radiant heat located just below the animal-testing surface (D'Amour, E. E. and Smith, D. L. J. Pharmacol. Exp. Ther. 1941 72:74-79). The light from the lamp projected through a small hole in the testing surface and was aimed at a photocell located 25 cm above the testing surface. A digital timer, connected to the apparatus, started when the heat source was activated. When the animal flicked its tail away from the heat source, the light from the projection lamp activated the photocell, simultaneously stopping the timer and turning off the lamp. The heat intensity was calibrated to result in baseline tail flick latencies of 2-3 seconds and a 10 second cutoff was used to minimize tissue damage.

### Example 4

### Nociceptive Testing

Nociceptive reflexes to a thermal stimulus were tested using the tail flick analgesia meter. This apparatus focuses a hot beam on the animal's tail. The time it takes for the rat to flick its tail away from the heat source is a measure of pain; the longer the animal leaves its tail on the hotspot, the greater the degree of pain relief. On the day prior to tail flick testing, animals were handled on the tail flick apparatus for 5-10 minutes to reduce stress-induced analgesia (Kelly, S. J. and Franklin, K. B., Neuropharmacology 1985 24:1019-1025; Terman et al., Science 1984 226:1270-1277). For single injection tested groups, animals were restrained in a small towel and a baseline tail flick latency was measured. Following the baseline measure, animals were given a drug injection and tail flick latencies were assessed every 10 minutes for 90 minutes. For the repeated injection groups, animals were given one drug injection every day for seven days. Prior to the first injection, a baseline tail flick latency was measured. Following this measure, animals were given a drug injection and tail flick latencies were assessed 10 minutes post-injection. This time point was selected because it is the point at which maximal WIN-induced antinociception is detected in this protocol. Post injection tail flick latencies were assessed on days 1, 3, 5, and 7. For all animals, tail flick latencies were converted into a percent of maximal possible effect (MPE) using the equation:
MPE=[(post-injection latency−baseline latency)/(10 s cutoff−baseline latency)]×100

### Example 5

### Tissue Sampling and Analysis

On the day following the last injection, animals were sedated with CO₂, and then decapitated. The brain and lumbar spinal cord were quickly extracted on ice, and a sample of the striatum, periaqueductal gray and dorsal horn was extracted, immersed in liquid nitrogen, and stored at −80° C. until immunoassays could be performed.

Co-immunoprecipitation experiments were performed on this tissue to determine the G-protein sub-type coupling profile of activated CB1 receptors. Tissue was bathed in vitro with either vehicle or cannabinoid receptor agonist and then solubilized. Samples were then divided and exposed to immobilized antibodies for Gαs, Gαi, or Gαo to isolate cannabinoid receptors bound to these G-protein sub-types. These samples were later subjected to western blotting using a specific CB1 antibody. Comparisons were made between tissue from animals previously treated chronically with vehicle, WIN 55 212-2, ultra-low dose SR 141716 or combination treatment of WIN and ultra-low dose SR 141716.

### Example 6

### Statistics

Separate two-way repeated measure ANOVAs were performed on the single injection groups, and repeated injection groups (see Example 2). Post-injection time (10-90 minutes for single injection groups) or day of injection (1-7 days for the repeated injection groups) were the within-subjects factors and drug group was the between-subjects factor. Whenever there were violations of sphericity, Huynh-Feldt corrections to the within-group degrees of freedom were reported. Because many drug group comparisons were irrelevant, a priori multiple comparisons were used to analyze the main drug effect using the Dunn's critical t-ratio. A corrected α of 0.005 was used for the single injection groups because there were 8 comparisons, and a corrected α of 0.01 was used for the repeated injection groups because there were 4 comparisons.

What is claimed is:

1. A composition comprising a cannabinoid receptor agonist in an amount effective to produce a therapeutic effect and a cannabinoid receptor antagonist in an amount effective to potentiate, but not antagonize, the therapeutic effect of the cannabinoid receptor agonist.

2. The composition of claim 1 wherein the cannabinoid receptor agonist is selected from the group consisting of endocannabinoids, (-)-trans-delta-9-tetrahydrocannabinol (delta-9-THC), CP-55,940, arachidonylethanolamide (anandamide), WIN 55 212-2, HU-210, HU-243, arachidonyl-2-chloroethylamide, arachidonylcyclopropylamide, O-1812, 2-arachidonoyl glycerol, dronabinol (marinol), sativex, cannabidiol, cannabinol, cannabichromene, cannabigerol, phytocannabinoids, endocannabinoid transporter inhibitors and cannabinoid receptor agonist metabolizing enzyme inhibitors.

3. The composition of claim 1 wherein the cannabinoid receptor antagonist is selected from the group consisting of SR 141716, SR 144528, AM-251, AM281 and LY320135.

4. The composition of claim 1 wherein the cannabinoid receptor agonist is delta-9-THC and the cannabinoid receptor antagonist is SR 141716.

5. The composition of claim 1 wherein the cannabinoid receptor agonist is WIN and the cannabinoid receptor antagonist is SR 141716.

6. The composition of claim 1 wherein the cannabinoid receptor agonist is cannabidiol and the cannabinoid receptor antagonist is SR 141716.

7. A method for potentiating a therapeutic effect of cannabinoid receptor agonist in a subject comprising administering a cannabinoid receptor agonist to the subject and administering a cannabinoid receptor antagonist to the subject in an amount effective to potentiate, but not antagonize, the therapeutic effect of the cannabinoid receptor agonist.

8. The method of claim 7 wherein the cannabinoid receptor agonist is selected from the group consisting of endocannabinoids, (-)-trans-delta-9-tetrahydrocannabinol (delta-9-THC), CP-55,940, arachidonylethanolamide (anandamide), WIN 55 212-2, HU-210, HU-243, arachidonyl-2-chloroethylamide, arachidonylcyclopropylamide, O-1812, 2-arachidonoyl glycerol, dronabinol (marinol), sativex, cannabidiol, cannabinol, cannabichromene, cannabigerol, phytocannabinoids, endocannabinoid transporter inhibitors and cannabinoid receptor agonist metabolizing enzyme inhibitors.

9. The method of claim 7 wherein the cannabinoid receptor antagonist is selected from the group consisting of SR 141716, SR 144528, AM-251, AM281 and LY320135.

10. The method of claim 7 wherein the therapeutic effect of the cannabinoid receptor agonist is potentiated without substantial undesirable side effects.

11. A method for inhibiting development of acute tolerance to a therapeutic action of a cannabinoid receptor agonist in a subject comprising administering the cannabinoid receptor agonist to the subject and administering a cannabinoid receptor antagonist to the subject in an amount effective to potentiate, but not antagonize, the therapeutic effect of the cannabinoid receptor agonist.

12. The method of claim 11 wherein the cannabinoid receptor agonist is selected from the group consisting of endocannabinoids, (-)-trans-delta-9-tetrahydrocannabinol (delta-9-THC), CP-55,940, arachidonylethanolamide (anandamide), WIN 55 212-2, HU-210, HU-243, arachidonyl-2-chloroethylamide, arachidonylcyclopropylamide, O-1812, 2-arachidonoyl glycerol, dronabinol (marinol), sativex, cannabidiol, cannabinol, cannabichromene, cannabigerol, phytocannabinoids, endocannabinoid transporter inhibitors and cannabinoid receptor agonist metabolizing enzyme inhibitors.

13. The method of claim 11 wherein the cannabinoid receptor antagonist is selected from the group consisting of SR 141716, SR 144528, AM-251, AM281 and LY320135.

14. A method for inhibiting development of chronic tolerance to a therapeutic action of a cannabinoid receptor agonist in a subject comprising administering the cannabinoid receptor agonist to the subject and administering a cannabinoid receptor antagonist to the subject in an amount effective to potentiate, but not antagonize the therapeutic effect of the cannabinoid receptor agonist.

15. The method of claim 14 wherein the cannabinoid receptor agonist is selected from the group consisting of endocannabinoids, (-)-trans-delta-9-tetrahydrocannabinol (delta-9-THC), CP-55,940, arachidonylethanolamide (anandamide), WIN 55 212-2, HU-210, HU-243, arachidonyl-2-chloroethylamide, arachidonylcyclopropylamide, O-1812, 2-arachidonoyl glycerol, dronabinol (marinol), sativex, cannabidiol, cannabinol, cannabichromene, cannabigerol, phytocannabinoids, endocannabinoid transporter inhibitors and cannabinoid receptor agonist metabolizing enzyme inhibitors.

16. The method of claim 14 wherein the cannabinoid receptor antagonist is selected from the group consisting of SR 141716, SR 144528, AM-251, AM281 and LY320135.

17. A method for reversing tolerance to a therapeutic action of a cannabinoid receptor agonist or restoring a therapeutic action of a cannabinoid receptor agonist in a subject comprising administering to the subject a cannabinoid receptor antagonist in an amount effective to potentiate, but not antagonize, the therapeutic effect of the cannabinoid receptor agonist.

18. The method of claim 17 wherein the cannabinoid receptor antagonist is selected from the group consisting of SR 141716, SR 144528, AM-251, AM281 and LY320135.

19. A method for treating a subject suffering from a condition treatable with a cannabinoid receptor agonist comprising administering a cannabinoid receptor agonist to the subject in an amount effective to produce a therapeutic effect and administering a cannabinoid receptor antagonist to the subject in an amount effective to potentiate, but not antagonize the therapeutic effect of the cannabinoid receptor agonist.

20. The method of claim 19 wherein the cannabinoid receptor agonist is selected from the group consisting of endocannabinoids, (-)-trans-delta-9-tetrahydrocannabinol (delta-9-THC), CP-55,940, arachidonylethanolamide (anandamide), WIN 55 212-2, HU-210, HU-243, arachidonyl-2-chloroethylamide, arachidonylcyclopropylamide, O-1812, 2-arachidonoyl glycerol, dronabinol (marinol), sativex, cannabidiol, cannabinol, cannabichromene, cannabigerol, phytocannabinoids, endocannabinoid transporter inhibitors and cannabinoid receptor agonist metabolizing enzyme inhibitors.

21. The method of claim 19 wherein the cannabinoid receptor antagonist is selected from the group consisting of SR 141716, SR 144528, AM-251, AM281 and LY320135.

22. The method of claim 19 wherein the subject is suffering from pain, nausea or vomiting, glaucoma, a movement disorder, neurodegeneration, anxiety, acute inflammation, chronic inflammation, pulmonary inflammation, hypertension, Alzheimer's disease, a gastrointestinal disorder, or atherosclerosis.

23. The method of claim 19 wherein the subject is suffering from acute post-surgical pain, obstetrical pain, acute or chronic inflammatory pain, pain associated with multiple sclerosis or cancer, pain associated with trauma, pain associated with migraines, neuropathic pain, central pain or chronic pain syndrome of a non-malignant origin.

24. The method of claim 19 wherein the subject is suffering from post-surgical pain, pain related to multiple sclerosis, pain related to cancer, or neuropathic pain.

25. A method for treating a subject suffering from a condition treatable with a cannabinoid receptor agonist comprising administering to a subject receiving cannabinoid receptor agonist therapy a cannabinoid receptor antagonist in an amount effective to potentiate, but not antagonize the therapeutic effect of the cannabinoid receptor agonist.

26. The method of claim 25 wherein the cannabinoid receptor antagonist is selected from the group consisting of SR 141716, SR 144528, AM-251, AM281 and LY320135.

27. The method of claim 25 wherein the subject is suffering from pain, nausea or vomiting, glaucoma, a movement disorder, neurodegeneration, anxiety, acute inflammation, chronic inflammation, pulmonary inflammation, hypertension, Alzheimer's disease, a gastrointestinal disorder, or atherosclerosis.

28. The method of claim 25 wherein the subject is suffering from acute post-surgical pain, obstetrical pain, acute or chronic inflammatory pain, pain associated with multiple sclerosis or cancer, pain associated with trauma, pain associated with migraines, neuropathic pain, central pain or chronic pain syndrome of a non-malignant origin.

29. The method of claim 25 wherein the subject is suffering from post-surgical pain, pain related to multiple sclerosis, pain related to cancer, or neuropathic pain.

30. The method of claim 25 wherein the subject is treated without substantial undesirable side effects.
